(19) United States
(12) Patent Application Publication
(10) Pub. No.: US 2021/0326518 A1
(43) Pub. Date: Oct. 21, 2021

(54) READING REPORT CREATING APPARATUS

(71) Applicant: Canon Medical Systems Corporation, Otawara-shi (JP)

(72) Inventors: Koichi TERAI, Shioya (JP); Takayuki TANEMOTO, Otawara (JP); Noriyuki HATAKEYAMA, Yokohama (JP); Takako MORITA, Nagoya (JP); Namiko SUDA, Nagoya (JP)

(73) Assignee: Canon Medical Systems Corporation, Otawara-shi (JP)

(21) Appl. No.: 17/231,452

(22) Filed: Apr. 15, 2021

(30) Foreign Application Priority Data
Apr. 17, 2020 (JP) 2020-074088

(51) Int. Cl. G06F 40/174; G16H 15/00

(52) U.S. Cl. CPC G06F 40/174; G16H 15/00

(57) ABSTRACT

According to one embodiment, a reading report creating apparatus includes a processing circuitry. The processing circuitry instructs saving of entry values of a plurality of entry items contained in a reading report creating screen. When saving is instructed, the processing circuitry extracts from the plurality of entry items a first entry item which requires entry of a value and in which a value has not been entered, and/or a second entry item in which an entry value is inconsistent with a specific entry item. The processing circuitry generates a displaying area in which the first entry item and/or the second entry item are/is arranged. The processing circuitry displays the displaying area on a display.

### CROSS-REFERENCE TO RELATED APPLICATIONS

This application is based upon and claims the benefit of priority from Japanese Patent Application No. 2020-074088, filed Apr. 17, 2020, the entire contents of which are incorporated herein by reference.

### FIELD

Embodiments described herein relate generally to a reading report creating apparatus.

### BACKGROUND

Reading reports are created based on medical images. For example, reading reports are created based on mammography images generated by a mammography apparatus.

### BRIEF DESCRIPTION OF THE DRAWINGS

FIG. 1 is a diagram showing a configuration example of a reading report creating apparatus according to a present embodiment.

FIG. 2 is a diagram showing an environmental example of creation by the reading report creating apparatus.

FIG. 3 is a diagram showing an example of a handwriting entry screen displayed on a tablet terminal.

FIG. 4 is a diagram showing an example of a reading report displaying screen displayed on a display.

FIG. 5 is a diagram showing a flow of creation processing of a reading report by the reading report creating apparatus.

FIG. 6 is a diagram showing an example of an extracted item displaying window for a finding item.

FIG. 7 is a diagram showing another example of the extracted item displaying window for a finding item.

FIG. 8 is a diagram showing an example of an extracted item displaying window for a comprehensive evaluation item.

FIG. 9 is a diagram showing another example of the extracted item displaying window for the comprehensive evaluation item.

FIG. 10 is a diagram schematically showing processing of judging consistency between a first reading and second reading according to an application example 1.

FIG. 11 is a diagram schematically showing processing of judging consistency between a current examination and a comparative examination according to an application example 2.

FIG. 12 is a diagram schematically showing an example of processing according to an application example 3.

FIG. 13 is a diagram schematically showing an example of processing according to an application example 4.

### DETAILED DESCRIPTION

In general, according to one embodiment, a reading report creating apparatus includes a processing circuitry. The processing circuitry instructs saving of entry values of a plurality of entry items contained in a reading report creating screen. When saving is instructed, the processing circuitry extracts from the plurality of entry items a first entry item which requires entry of a value and in which a value has not been entered, and/or a second entry item in which an entry value is inconsistent with a specific entry item. The processing circuitry generates a displaying area in which the first entry item and/or the second entry item are/is arranged. The processing circuitry displays the displaying area on a display.

Hereinafter, embodiments of the reading report creating apparatus will be described in detail with reference to the accompanying drawings.

A reading report creating apparatus 100 according to an embodiment is a computer for an operator to create a reading report. The reading report creating apparatus 100 communicates with a server (not shown) that manages various types of information for creating a reading report, and executes creation of a reading report. Specifically, for example, upon receipt of reading order information and patient information, the server updates a management list. The management list is a list for managing whether or not creation of a reading report has been performed in response to the reading order. The reading report creating apparatus 100 obtains a reading order not executed, and creates a reading report regarding this reading order. The completed reading report is transmitted to the server. In this manner, the management list of the server is updated. An operator is assumed to be a reader; however, other medical staff such as other doctors, nurses, technicians, etc., or patients and other third parties are not excluded.

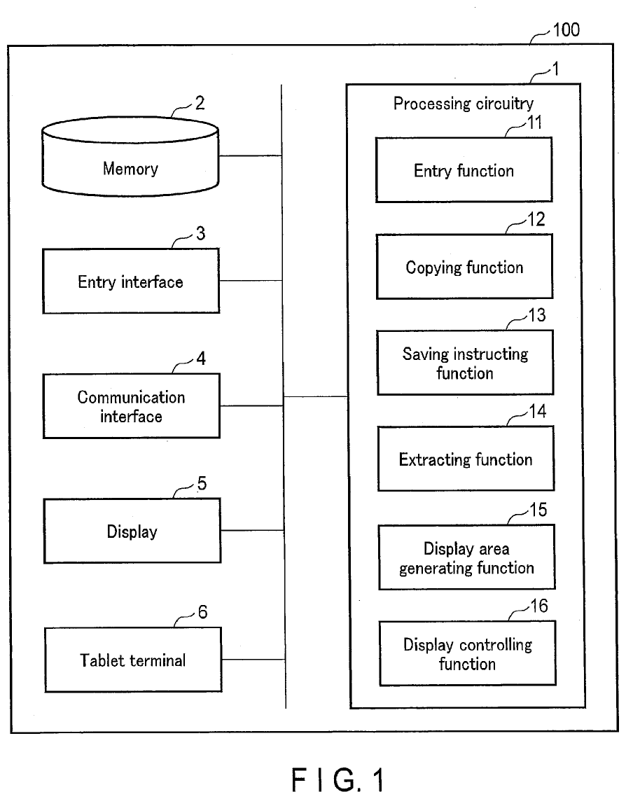

FIG. 1 is a diagram showing a configuration example of a reading report creating apparatus 100 according to a present embodiment. As shown in FIG. 1, the reading report creating apparatus 100 includes a processing circuitry 1, a memory 2, an entry interface 3, a communication interface 4, a display 5, and a tablet terminal 6.

The processing circuitry 1 includes a processor such as a central processing unit (CPU), a graphics processing unit (GPU), etc. In order to create a reading report, by a processor of the processing circuitry 1 activating an reading report creating program installed in the memory 2, etc., the processing circuitry 1 implements an entry function 11, a copying function 12, a saving instructing function 13, an extracting function 14, a displaying area generating function 15, a display controlling function 16, etc. The functions 11 to 16 are not necessarily implemented by a single processing circuitry. A processing circuitry may be configured by combining a plurality of independent processors that execute respective programs to implement the respective functions 11 to 16. The entry function 11, the copying function 12, the saving instructing function 13, the extracting function 14, the displaying area generating function 15, and the display controlling function 16 may be a modularized program that constitutes a reading report creating program, or may be individual programs. These programs are stored in the memory 2.

The entry function 11 enters values in a plurality of entry items contained in the reading report creating screen. The entry function 11 may enter values in accordance with instructions by a reader via the entry interface 3 or the tablet terminal 6, or may enter values determined in accordance with a predetermined algorithm. The entry items refer to all items that can be entered in the reading report creating screen, for example, “finding attribute”, “category”, “comparative reading”, “schema”, “key image”, and so on. The values in the entry items refer to all types of information that can be entered in the entry items, for example, a phrase such as “diffuse”, “scattered”, etc., for the entry item “finding attribute”, a numerical value such as “3”, “5”, etc., or a symbol for the entry item “category”, a fixed phrase such as “present”, “absent”, etc., for the entry item “comparative reading”, a lesion site entered by handwriting for the entry item “schema”, an attached key image for the entry item “key image”, and so on.

The copying function 12 copies, to an entry item which is an entry target, an entry value of another entry item, in accordance with a copy instruction via the entry interface 3 or the tablet terminal 6.

The saving instructing function 13 instructs saving of an entry value of an entry item in accordance with a copy instruction via the entry interface 3 or the tablet terminal 6. The entry value for which saving is instructed is saved in the memory 2.

When saving is instructed via the saving instructing function 13, the extracting function 14 extracts, from a plurality of entry items, a first entry item which requires entry of a value and in which a value has not been entered, and/or a second entry item in which an entry value is inconsistent with a specific entry item. Hereinafter, the extracted first entry item will be referred to as an unentered item, and the extracted second entry item will be referred to as an inconsistent item.

The displaying area generating function 15 generates a displaying area in which the unentered item and/or the inconsistent item extracted by the extracting function 14 are/is arranged. Hereinafter, the generated displaying area will be referred to as an extracted item displaying window.

The display controlling function 16 displays various types of information on the display 5 and the tablet terminal 6. For example, the display controlling function 16 displays the reading report creating screen or the extracted item displaying window on the display 5 or the tablet terminal 6.

The memory 2 is a storage device such as a read only memory (ROM), a random access memory (RAM), a hard disk drive (HDD), a solid state drive (SSD), an integrated circuit storage device, etc., which stores various types of information. Other than these storage devices, the memory 2 may be a driving device which reads and writes various types of information from and to, e.g., a portable storage medium such as a compact disc (CD), a digital versatile disc (DVD), or a flash memory, or a semiconductor memory device. Furthermore, the memory 2 may be contained in another computer coupled to the reading report creating apparatus 100 via a network.

The entry interface 3 receives various operations entered by a reader, converts the received operations into electric signals, and outputs them to the processing circuitry 1. Specifically, the entry interface 3 is coupled to entry devices such as a mouse, a keyboard, a track ball, a switch, buttons, a joystick, a touch pad, a touch panel display, and the like. The entry interface 3 outputs to the processing circuitry 1 the electric signals corresponding to the operations entered in the entry device. Furthermore, the entry device connected to the entry interface 3 may be an entry device provided in a separate computer connected via a network or the like.

The communication interface 4 is an interface for data-communicating various types of medical information with another computer such as a hospital information system (HIS), a radiology information system (RIS), a picture archiving and communication system (PACS), a medical image diagnostic apparatus, etc. For example, the communication interface 4 receives medical images from a medical image diagnostic apparatus or a PACS.

The display 5 displays various types of information in accordance with the display controlling function 16 of the processing circuitry 1. For example, the display 5 displays thereon the reading report creating screen or the extracted item displaying window. As display 5, a liquid crystal display (LCD), a cathode ray tube (CRT) display, an organic electro luminescence display (OELD), a plasma display, or other discretionary display may be used as appropriate. Furthermore, a projector may be provided instead of or in addition to the display 5.

The tablet terminal 6 is a portable computer having a display on which a touch panel is mounted. The tablet terminal 6 displays various types of information on its display in accordance with the display controlling function 16 of the processing circuitry 1. For example, the display 5 displays thereon the reading report creating screen or the extracted item displaying window. The tablet terminal 6 receives various operations entered by a reader via the touch panel, converts the received operations into electric signals, and outputs them to the processing circuitry 1. As a substitute for the tablet terminal 6, a smart phone or a notebook computer may be used.

Hereinafter, an operation example of the reading report creating apparatus 100 will be described. By way of example, the following description of the embodiments will assume creation of a reading report in a mammography examination. However, a reading report according to the embodiments may target any body part. As a medical image to be interpreted, any kind of medical image taken by any medical image diagnostic apparatus is applicable.

In a mammography examination, each of the right and left breasts of an examinee is X-rayed in an imaging direction such as a mediolateral oblique view (MLO) or a craniocaudal view (CC) by a mammography apparatus, and an X-ray image of the right breast and an X-ray image of the left breast are generated. A reader interprets the X-ray images of the right breast and the left breast, thereby creating a reading report. The X-ray images of breasts are also called mammography images.

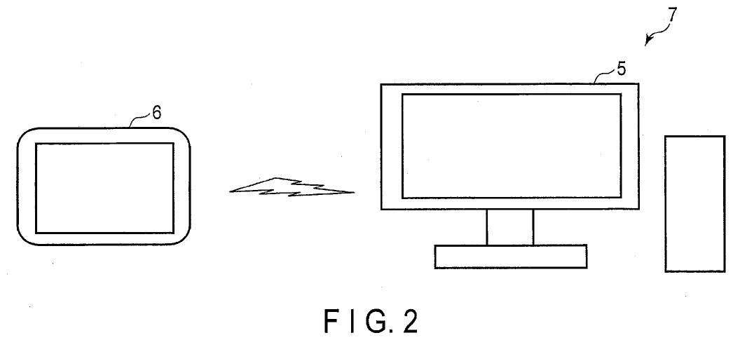

FIG. 2 is a diagram showing an environmental example of creation by the reading report creating apparatus 100. As shown in FIG. 2, the reading report creating apparatus 100 includes the tablet terminal 6 and a desktop personal computer (PC) 7. The desktop PC 7 includes the processing circuitry 1, the memory 2, the entry interface 3, the communication interface 4, and the display 5. Various types of information are transmitted and received between the tablet terminal 6 and the desktop PC 7 via wire or wirelessly. A reading report creating screen is displayed on the tablet terminal 6 and the desktop PC 7. The reading report creating screen according to the embodiment includes a handwriting entry screen and a reading report displaying screen. The handwriting entry screen is displayed on the tablet terminal 6, and the reading report displaying screen which is a body screen is displayed on the desktop PC 7. A reader such as a radiologist enters by handwriting an entry item regarding each finding via the tablet terminal 6, conducts reading on a mammography image or checking on a reading report by using the desktop PC7, and creates the reading report. The reading reports are sequentially created one by one for each of the examinees subjected to a mammography examination.

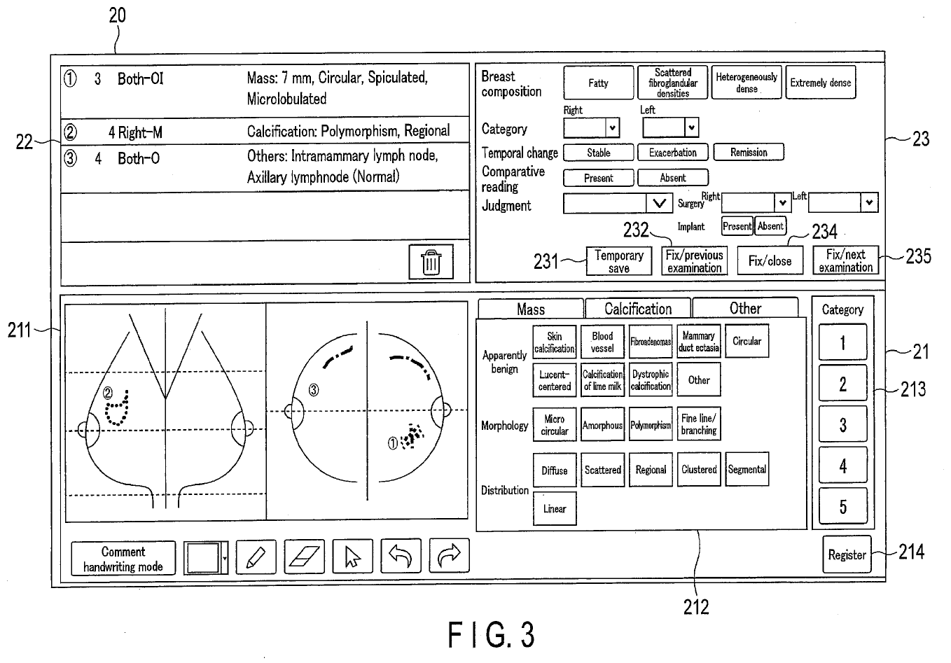

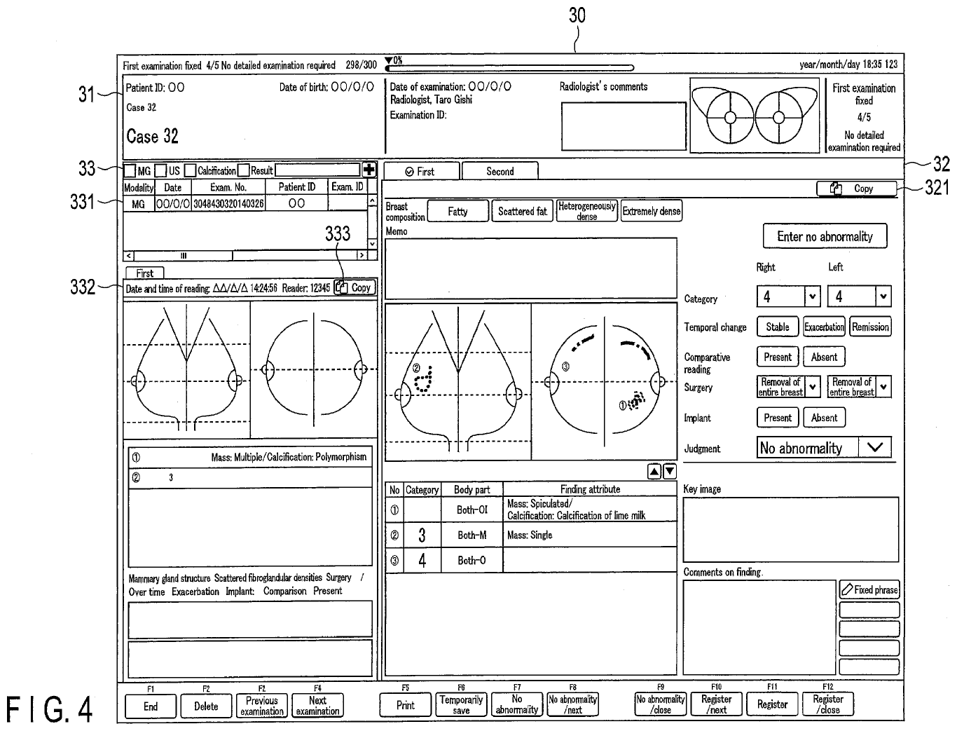

FIG. 3 is a diagram showing an example of a handwriting entry screen 20 displayed on the tablet terminal 6. FIG. 4 is a diagram showing an example of the reading report displaying screen 30 displayed on the display 5. The handwriting entry screen 20 is a displaying area for entry of findings regarding an examinee designated in the reading report displaying screen 30. The reading report displaying screen 30 is a main screen of the reading report creating screen and is a displaying area for a reading report on each examinee.

As shown in FIG. 3, the handwriting entry screen 20 includes a finding entry window 21, a finding displaying window 22, and a comprehensive evaluation entry window 23. As shown in FIG. 3, the finding entry window 21 displays thereon various entry items regarding findings (hereinafter, referred to as finding items) and their entry values. Examples of a finding item include “schema”, “finding attribute”, and “category”. Specifically, the finding entry window 21 includes a schema displaying area 211, a finding entry area 212, a category entry area 213, and a register button 214. The schema displaying area 211 displays thereon a schema of the right breast and the left breast which are examination objects. On the schema, an annotation of a lesion site is drawn in a free entry format via the touch panel of the tablet terminal 6. Annotations are preferably displayed in such a manner as to be visually distinguishable by color, line type, etc., for each lesion. In the finding entry area 212, GUI buttons corresponding to fixed phrases for finding attributes are displayed. The finding attributes are classified according to the type of lesion, such as “tumor”, “calcification”, etc. The finding attributes are further classified according to a perspective such as a feature amount of each lesion, and fixed phrases corresponding to typical evaluations for each perspective are prepared. For example, in the case of lesion “calcification”, it is divided into the perspectives “typically benign”, “morphology”, and “distribution”. As another example, in the case of the perspective “distribution”, GUI buttons are respectively displayed for evaluations such as “diffuse”, “scattered”, “regional”, “clustered”, “segmental”, “linear”, etc. In the category entry area 213, a GUI button corresponding to a category is displayed. As the category, a category defined by any institution may be used. For example, the category of this mammography guideline is divided into five stages from “1” to “5”. The register button 214 is a GUI button for instructing saving of an entry value entered in the finding entry window 21.

As shown in FIG. 3, the finding displaying window 22 displays, for each lesion, findings corresponding to entry values entered in the finding items entered in the finding entry window 21. For example, a category, a lesion position, and a finding attribute are displayed for each lesion. For example, the second lesion site corresponds to category “4”, is present in area “M” of the right breast, and corresponds to disease type “calcification”, morphology “polymorphism”, and distribution “regional”. Lesion sites of the breasts are categorized into the right breast, both breasts, and the left breast. A displaying area of a numerical value or a symbol (hereinafter referred to as a category value) of a category is divided into the right, the center, and the left corresponding to the right breast, both breasts, and the left breast, respectively. The category value is displayed in a displaying area corresponding to a position of the corresponding lesion site. For example, the second lesion site is positioned in the right breast. Thus, category value “4” is displayed on the right side in the category displaying area. As described in the above, displaying a category value at a position corresponding to the lesion site in the category displaying area enables a reader to intuitively ascertain the position of the lesion site only by checking the category value.

As shown in FIG. 3, the comprehensive evaluation entry window 23 displays thereon entry items regarding a comprehensive evaluation on the entirety of the breasts (hereinafter, referred to as comprehensive evaluation items) and their entry values. Examples of the comprehensive evaluation entry items include “breast composition”, “category”, “temporal change”, “comparative reading”, “judgment”, “surgery”, and “implant”. In the comprehensive evaluation entry window 23, fixed phrases are displayed through the use of a GUI button or a pull-down menu for each entry item regarding a comprehensive evaluation. In the comprehensive evaluation entry window 23, a temporary save button 231, a fix/previous examination button 232, a fix/close button 233, and a fix/next examination button 235 are displayed. The temporary save button 231 is a GUI button configured to instruct temporary saving of an entry value entered in the comprehensive evaluation entry window 23. The fix/previous examination button 232 is a GUI button configured to instruct saving of an entry value entered in the comprehensive evaluation entry window 23 and to instruct switching of the handwriting entry screen 20 from a screen of a current examination to a screen of a previous examination corresponding to an examination number one before. The fix/close button 233 is a GUI button configured to instruct saving of an entry value entered in the comprehensive evaluation entry window 23, and to instruct ending of displaying of the handwriting entry screen 20. The fix/next examination button 235 is a GUI button configured to instruct saving of an entry value entered in the comprehensive evaluation entry window 23 and to instruct switching of the handwriting entry screen 20 from a screen of a current examination to a screen of a next examination corresponding to an examination number one after.

As shown in FIG. 4, the reading report displaying screen 30 includes a basic information displaying area 31, a current examination displaying area 32, a past examination displaying area 33, and the like. In the basic information displaying area 31, patient information on an examinee of a current examination and examination information are displayed. The current examination means a mammography examination which is a target for creation of a reading report. In the current examination displaying area 32, a reading report created via the handwriting entry screen 20 using the tablet terminal 6 is displayed. In the past examination displaying area 33, a reading report on a past examination for an examinee is displayed. The past examination displaying area 33 includes a past examination list 331 and a past report displaying area 332. In the past examination list 331, a list of past examinations conducted on an examinee is displayed. In the past report displaying area 332, a reading report on a single past examination selected from the past examination list 331 is displayed. In the current examination displaying area 32, a copy button 321 is further displayed. In the past examination displaying area 33, a copy button 333 is further displayed. The copy button 321 is a GUI button for copying an entry value of a predetermined entry item for first reading to a corresponding predetermined entry item for second reading. The copy button 333 is a GUI button for copying an entry value of a predetermined entry item of an reading report on a single past examination selected from the past examination list 331 to a corresponding predetermined entry item for a current examination.

Next, creation processing of a reading report by the reading report creating apparatus 100 will be described.

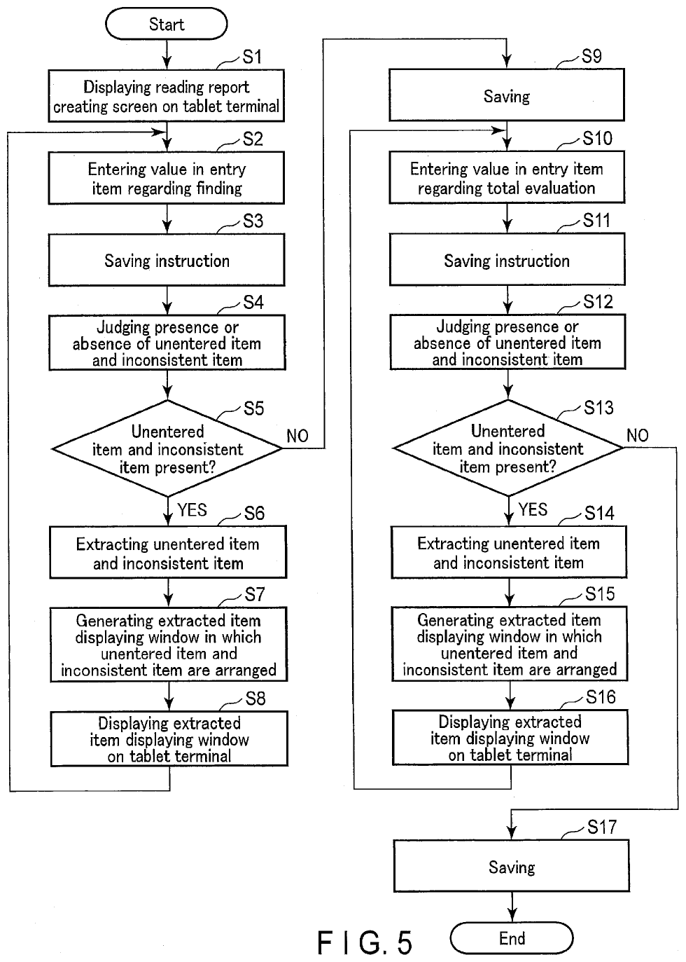

FIG. 5 is a diagram showing a flow of creation processing of a reading report by the reading report creating apparatus 100.

First, the processing circuitry 1 implements the display controlling function 16, thereby displaying the reading report creating screen on the tablet terminal 6 (step S1). More specifically, the processing circuitry 1 displays the handwriting entry screen 20 shown in FIG. 3 on the tablet terminal 6, and displays the reading report creating screen 30 on the display 5.

After step S1, the processing circuitry 1 implements the entry function 11, thereby entering a value in an entry item regarding a finding (finding item) (step S2). More specifically, the processing circuitry 1 enters a value in the finding item in accordance with an instruction from a reader via the tablet terminal 6. For example, the reader performs an entry of a value in the finding item as described below. First, an operation of drawing an annotation in such a manner as to be superimposed on a schema is performed, via the touch panel, on the schema displaying area 211 of the finding entry window 21 of the handwriting entry screen 20 shown in FIG. 3. An annotation is drawn by freehand. In response to this operation, the processing circuitry 1 enters, as a value, a lesion site indicated by the annotation in the finding item “schema”. Next, the reader enters a finding attribute regarding the lesion site entered in the schema. Specifically, the reader operates, via the touch panel, a GUI button of a fixed phrase for a finding attribute displayed in the finding entry area 212 shown in FIG. 3. In response to this operation, the processing circuitry 1 enters, as a value, a designated fixed phrase for the finding attribute in the finding item “finding attribute”. Next, the reader enters a category regarding the lesion site entered in the schema and the finding attribute. Specifically, the reader performs an operation of designating, via the touch panel, a GUI button of a category value displayed in the category entry area 123 shown in FIG. 3. In response to this operation, the processing circuitry 1 enters a designated category value in the finding item “category”. Through the display controlling function 16, an entry value of each finding item is displayed on the handwriting entry screen 20.

The above entry in the finding attribute is one example. A sentence about a finding may be entered in, for example, a free entry format. Drawing of annotation on the schema may be entered using a fixed stamp prepared in advance.

After step S2, the processing circuitry 1 implements the saving instructing function 13, thereby instructing saving (step S3). Upon the completion of the entering operation on finding items in step S2, the reader performs an operation of pressing the register button 214 shown in FIG. 3 via the touch panel. The processing circuitry 1 receives, as a saving instruction, a command in accordance with this operation from the tablet terminal 6.

When saving is instructed in step S3, the processing circuitry 1 implements the extracting function 14, thereby judging the presence or absence of an unentered item and/or inconsistent item from among the finding items displayed on the handwriting entry screen 20 (step S4). Hereinafter, processing regarding an unentered item and processing regarding an inconsistent item will be described.

First, processing regarding an unentered item will be described. The plurality of finding items displayed on the handwriting entry screen 20 are preset to either a mandatory entry item or an optional entry item. With respect to the finding items displayed on the handwriting entry screen 20, for example, the category is set to the mandatory entry item, and the schema and the finding attribute are set to the optional entry item. In step S4, the processing circuitry 1 judges, from among the mandatory entry items displayed on the handwriting entry screen 20, that a finding item in which a value has not been entered is an unentered item. Even in the case where a value has not been entered in a finding item, if it is an optional entry item, this particular finding item may not be judged as an unentered item.

Next, processing regarding an inconsistent item will be described. For each respective one of the finding items displayed on the handwriting entry screen 20, another finding item (hereinafter, referred to as a consistency required item) required to have consistency with the respective one finding item (hereinafter, referred to as a consistency judgment item) and a consistency fulfillment condition are set. The inconsistency fulfillment condition is specified in a relationship between an entry value of the consistency judgment item and an entry value of the consistency required item, and this relationship is required to make a determination that there is consistency between these entry values from various perspectives such as a logical perspective and a medical perspective. Consistency is determined according to a property of a consistency judgment item, a property of a consistency required item, and a combination of the consistency judgment item and the consistency required item. For example, the consistency judgment item, the consistency required item, and the consistency fulfillment condition are registered in a Lookup Table (LUT) in such a manner that they are correlated with each other. Hereinafter, the aforementioned LUT will be referred to as a consistency judgment table. The consistency judgment table is stored in the memory 2, etc. The processing circuitry 1 reads an entry value of the consistency required item that is correlated with the consistency judgment item in the consistency judgment table. The processing circuitry 1 reads the consistency fulfillment condition that is correlated with the consistency judgment item in the consistency judgment table. The processing circuitry 1 then judges whether or not an entry value of the consistency judgment item and the entry value of the consistency required item fulfill the consistency fulfillment condition. In the case where these entry values fulfill the consistency fulfillment condition, the consistency judgment item and the consistency required item are not extracted as inconsistent items. In the case where these entry values do not fulfill the consistency fulfillment condition, these items are extracted inconsistent items.

For example, a category value of 2 or more indicates that a corresponding lesion site exhibits a finding attribute of some kind. Therefore, in the case where an entry value of the finding item “category” is “2” or more, a value should be entered in the finding item “finding attribute”. In this case, for example, the consistency judgment item is set to “finding attribute”, the consistency required item is set to “category”, and the consistency fulfillment condition is set to “if a category value is 2 or more, the finding attribute has a value entered”. In contrast, for example, the consistency judgment item may be set to “category”, the consistency required item may be set to “finding attribute”, and the consistency fulfillment condition may be set to “if the finding attribute has no value entered, a category value is equal to 1”. For example, in the case where the finding item “finding attribute” has no value entered with the finding item “category” having the entry value of “4”, the finding items “category” and “finding attribute” are judged as inconsistent items. The consistency judgment item and the consistency required item are examples of an inconsistent item.

After step S4, the processing circuitry 1 implements the extracting function 14, thereby judging whether an unentered item and/or an inconsistent item are/is present or absent (step S5). In the case where it is judged in step S5 that at least one unentered item or inconsistent item is present, the processing circuitry 1 judges that an unentered item and/or an inconsistent item are/is present. If it is judged in step S5 neither an unentered item nor an inconsistent item is present, the processing circuitry 1 judges that an unentered item and/or an inconsistent item are/is absent.

If it is judged in step S5 that an unentered item and/or an inconsistent item are/is present (step S5: Yes), the processing circuitry 1 implements the extracting function 14, thereby extracting the unentered item and/or the inconsistent item judged in step S4 (step S6).

After step S6, the processing circuitry 1 implements the displaying area generating function 15, thereby generating a displaying window (extracted item displaying window) in which the unentered item and/or the inconsistent item extracted in step S6 are/is arranged (step S7). After step S7, the processing circuitry 1 implements the display controlling function 16, thereby displaying, on the tablet terminal 6, the extracted item displaying window generated in step S7 (step S8).

Herein, steps S7 and S8 will be described in detail. The extracted item displaying window for the finding items is generated by arranging, in a single displaying window, all of the unentered items and/or inconsistent items extracted in step S6. Specifically, the extracted item displaying window is generated by arranging a GUI component of an unentered item and/or inconsistent item in the displaying window. The processing circuitry 1 may display the extracted item displaying window in such a manner as to be superimposed on the handwriting entry screen 20 or may display the extracted item displaying window by erasing the handwriting entry screen 20.

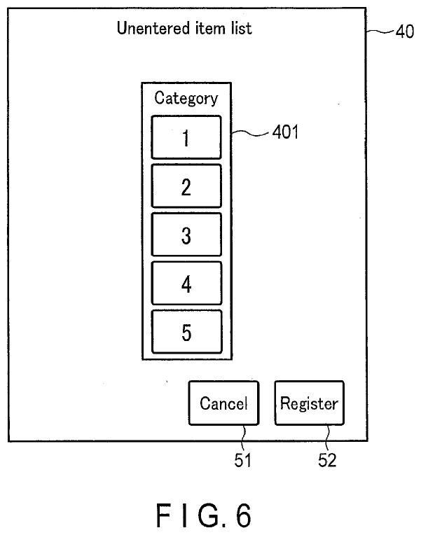

FIG. 6 is a diagram showing an example of the extracted item displaying window 40. FIG. 6 shows, as an example, the extracted item displaying window 40 in the case where the unentered item “category” is extracted in step S6. In this case, the GUI button 401 of the unentered item “category” is arranged in the extracted item displaying window 40. The GUI button 401 is composed of GUI buttons that can be pressed via a touch panel, etc., and respectively correspond to category values. In the extracted item displaying window 40 shown in FIG. 6, only the GUI button 401 of the unentered item “category” is arranged, and GUI buttons of the entered items “schema”, “finding attribute”, etc., are not arranged. By displaying the extracted item displaying window 40 in which only the unentered item is arranged, a reader can notice accidental omission of entry of a value in a mandatory entry item, and the processing circuitry 1 can create a reading report free from accidental omission of entry of a value in a mandatory entry item.

As shown in FIG. 6, in the extracted item displaying window 40 for the finding items, a cancel button 51 and a register button 52 are arranged. In the case where the cancel button 51 is pressed, the processing circuitry 1 deletes the extracted item displaying window 40. In this case, a saving instruction entered in step S3 is canceled, and the handwriting entry screen 20 is displayed. As in step S2, the reader performs an entry operation of a value in an unentered mandatory entry item via the handwriting entry screen 20. In the case where the register button 52 is pressed, as in step S3, the processing circuitry 1 implements the saving instructing function 13, thereby instructing saving of a value entered in the unentered item displayed on the extracted item displaying window 40.

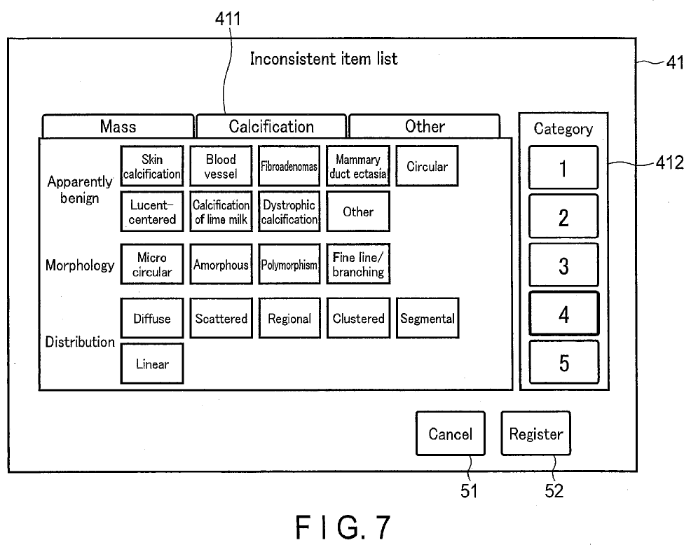

FIG. 7 is a diagram showing, as another example, the extracted item displaying window 41 for the finding items. FIG. 7 shows, as an example, the extracted item displaying window 41 in the case where the inconsistent items “finding attribute” and “category” are extracted in step S6. More specifically, FIG. 7 shows the case in which no entry is made to the finding item “finding attribute” in spite of the condition in which an entry value of the finding item “category” is “4”. In this case, the finding items “finding attribute” and “category” are extracted as inconsistent items as described above.

As shown in FIG. 7, in the extracted item displaying window 41, a GUI button 411 of the inconsistent item “finding attribute”, a GUI button 412 of the inconsistent item “category”, the cancel button 51, and the register button 52 are arranged. To make it easy for a reader to check inconsistency, the GUI button 411 and the GUI button 412 respectively corresponding to the inconsistent items “finding attribute” and “category” are displayed side by side. In the case where a value is already entered before saving is instructed, the entered value is preferably displayed along with the inconsistent items. Among GUI buttons included in the GUI button 412 of “category”, a GUI button corresponding to the value “4” entered before a saving instruction is emphasized. By displaying the extracted item displaying window 41 in which only the inconsistent items are arranged, a reader can notice inconsistency in values between the finding items, and the processing circuitry 1 can create a reading report free from inconsistency.

The extracted item displaying windows described in the above are merely examples, and are not limitations. For example, in the case where the unentered item or the inconsistent item is “schema”, a GUI component of “schema” is arranged in the extracted item displaying window. Furthermore, in the case where the unentered item and the inconsistent item are extracted, the GUI button of the unentered item and the GUI button of the inconsistent item may be arranged in a single extracted item displaying window.

After step S8, the processing circuitry 1 returns to step S2, enters values in the unentered item and the inconsistent item displayed on the extracted item displaying window, and repeats steps S2 to S8 until it is no longer judged in step S5 that the unentered item and/or the inconsistent item are/is present.

If it is judged in step S5 that the unentered item and/or the inconsistent item are/is absent (step S5: No), the processing circuitry 1 implements the saving instructing function 13, thereby saving, in the memory 2, an entry value entered in the finding item in step S2 (step S9). The memory 2 stores entry values of the finding items for each lesion site along with patient information and examination information.

Steps S2 to S8 are performed for each lesion site. For example, a reader draws an annotation of a single lesion site on the schema, enters a finding attribute and a category value for this lesion site, and presses the register button 214. In the case where saving of entry values is performed, the reader draws an annotation of a next lesion site, enters a finding attribute and a category for this next lesion site, and presses the register button 214. In this manner, the finding is entered for all of the lesion sites.

After step S9, the processing circuitry 1 implements the entry function 11, thereby entering a value in the input item regarding a comprehensive evaluation (comprehensive evaluation item) (step S10). More specifically, the processing circuitry 1 enters a value in the comprehensive evaluation item in accordance with an instruction from a reader via the tablet terminal 6. For example, the reader performs an entry operation of a value in the comprehensive evaluation item as will be described below. First, the reader designates an entry value by operating, via the touch panel, GUI components such as a GUI button and a pull-down menu for comprehensive evaluation items such as “breast composition”, “category”, “temporal change”, “comparative reading”, “judgment”, “surgery”, and “implant”, which are displayed on the comprehensive evaluation entry window 23 of the handwriting entry screen 20 shown in FIG. 3. In response to this operation, the processing circuitry 1 enters a designated value in the comprehensive evaluation item.

After step S10, the processing circuitry 1 implements the saving instructing function 13, thereby instructing saving (step S11). Upon completion of the entry operation on the finding items in step S10, the reader performs an operation of pressing, via the touch panel, the fix/previous examination button 232, the fix/close button 234, and the fix/next examination button 235 shown in FIG. 3. The processing circuitry 1 receives, as a saving instruction, a command in accordance with this operation from the tablet terminal 6.

When saving is instructed in step S11, the processing circuitry 1 implements the extracting function 14, thereby judging the presence or absence of the unentered item and/or the inconsistent item in the comprehensive evaluation entry window 23 (step S12). The judgment as to the presence or absence of the unentered item and/or the inconsistent item regarding the comprehensive evaluation items is similar to the judgment as to the presence or absence of the unentered item and/or the inconsistent item regarding the finding items. Each of the comprehensive evaluation items is preset to either the mandatory entry item or the optional entry item. For example, the comprehensive evaluation items “category” and “judgment” are set to the mandatory entry items. In the case where no value is entered in the mandatory entry item, a judgment is made that this particular entry item corresponds to the unentered item. A conditional mandatory entry item may be set. For example, “temporal change” is an optional entry item if a value of “comparative reading” indicates “absent”; however, it is a mandatory entry item if a value of “comparative reading” indicates “present”. For the comprehensive evaluation items also, a consistency judgment table in which a consistency judgment item, a consistency required item, and a consistency fulfillment condition are registered in such a manner that they are correlated with each other is created in advance and is stored in the memory 2, etc. For example, the consistency judgment item “comparative reading”, the consistency required item “temporal change”, and the consistency fulfillment condition “if a value of comparative reading indicates ‘present’, a value indicative of stable, a value indicative of exacerbation, or a value indicative of remission is entered in ‘temporal change’” are registered in the consistency judgment table. In the case where no key image is attached to an reading report in spite of the condition in which a category value is a predetermined level (for example, 4) or more, a total evaluation item “key image” may be extracted as an unentered item or an inconsistent item.

After step S12, the processing circuitry 1 implements the extracting function 14, thereby judging whether an unentered item and/or an inconsistent item are/is present or absent (step S13). If it is judged in step S12 that the unentered item and/or the inconsistent item are/is present (step S13: Yes), the processing circuitry 1 implements the extracting function 14, thereby extracting the unentered item and/or the inconsistent item (step S14). After step S14, the processing circuitry 1 implements the displaying area generating function 15, thereby generating a displaying window (extracted item displaying window) in which the unentered item and/or the inconsistent item extracted in step S14 are/is arranged (step S15).

Herein, steps S14 and S15 will be described in detail. The extracted item displaying window for a comprehensive evaluation is generated by arranging, in a single displaying window, all of the unentered items and/or inconsistent items extracted in step S14. Specifically, the extracted item displaying window is generated by arranging a GUI component of an unentered item and/or inconsistent item in the displaying window. The processing circuitry 1 may display the extracted item displaying window for a comprehensive evaluation in such a manner as to be superimposed on the handwriting entry screen 20 or may display the extracted item displaying window by erasing the handwriting entry screen 20.

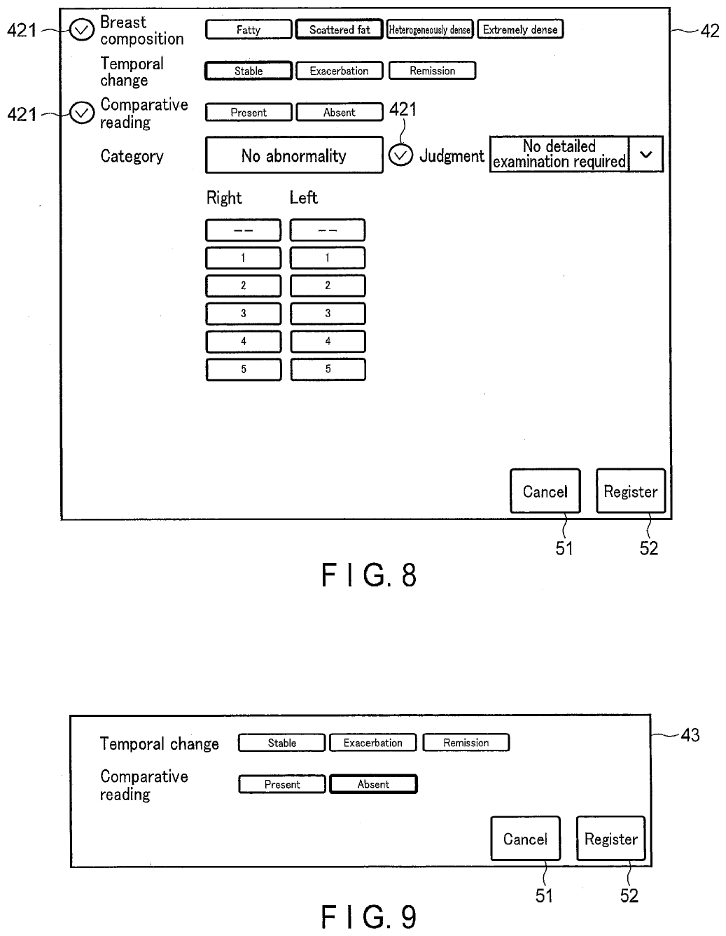

FIG. 8 is a diagram showing an example of an extracted item displaying window 42 for a comprehensive evaluation item. FIG. 8 shows, as an example, the extracted item displaying window 42 in the case where the unentered items “category” and “temporal change” are extracted in step S13. In this case, a GUI button of the unentered item “temporal change” and a GUI button of the unentered item “category” are arranged in the extracted item displaying window 42. An entered entry item (hereinafter, referred to as a reference item) relating to this unentered item is preferably displayed. For example, if reference items with respect to the unentered item “temporal change” are “breast composition” and “comparative reading”, the GUI button of the reference item “breast composition” and the GUI button of the reference item “comparative reading” are displayed in a line with the GUI button of the unentered item “temporal change”. An entered entry item is displayed along with an unentered item, so that a reader can make an entry free from inconsistency with the entered entry item. The entered entry item (reference item) is preferably displayed in such a manner as to be visually distinguishable from the unentered item. For example, as shown in FIG. 8, GUI components of the reference item “breast composition” and the reference item “comparative reading” are preferably assigned marks 421 each indicating that an entry has been made. The reference items and the marks 421 may be displayed in the extracted item displaying window 41 for the finding items.

FIG. 9 is a diagram showing, as another example, an extracted item displaying window 43 for the comprehensive evaluation item. FIG. 9 shows, as an example, the extracted item displaying window 43 in the case where the inconsistent items “temporal change” and “comparative reading” are extracted in step S13. In this case, GUI components respectively corresponding to the inconsistent items “temporal change” and “comparative reading” are arranged in the extracted item displaying window 43. As in the case with the finding item, for the comprehensive evaluation item, the consistency judgment item and the consistency fulfillment condition are set. For example, the consistency fulfillment condition is set to “if a value of the comparative reading indicates ‘absent’, no value is entered in ‘temporal change’”, “if a value of the comparative reading indicates ‘present’, a value is entered in ‘temporal change’”, or the like.

After step S15, the processing circuitry 1 implements the display controlling function 16, thereby displaying, on the tablet terminal 6, the extracted item displaying window generated in S15 (step S16).

After step S16, the processing circuitry 1 returns to step S11, enters values in the unentered item and the inconsistent item displayed on the extracted item displaying window for the comprehensive evaluation item, and repeats steps S11 to S16 until it is no longer judged in step S13 that the unentered item and/or the inconsistent item are/is present.

If it is not judged in step S13 that the unentered item and/or the inconsistent item are/is present (step S13: No), the processing circuitry 1 implements the saving instructing function 13, thereby saving, in the memory 2, an entry value of the comprehensive evaluation item (step S17). The memory 2 stores the entry value of the comprehensive evaluation item along with patient information and examination information. Accordingly, a reading report on an examinee concerned is completed.

In this manner, creation processing of a reading report by the reading report creating apparatus 100 is terminated.

The above flow of creation processing of a reading report is one example and may be modified in various ways. For example, the reading report creating screen described in the above is composed of the two screens including the handwriting entry screen 20 and the reading report displaying screen 30; however, the handwriting entry screen 20 and the reading report displaying screen 30 may form one screen or three or more screens.

In the extracted item displaying window described in the above, all of the unentered items and/or inconsistent items are arranged; however, these items may be arranged in such a manner that they are divided into two or more extracted item displaying windows, and may be displayed in parallel or in sequence.

Entry of values in the finding item and the total evaluation item may be automatically performed by pressing the button “enter no abnormality” shown in FIG. 4. In the case where the button “enter no abnormality” is pressed, a preset value corresponding to no abnormality is automatically entered in each of the finding items and total evaluation items. This saves a reader time and trouble in making an entry operation. Furthermore, entry of a preset value enables reduced variation in entry value and improved quality of a reading report.

According to the embodiment described in the above, in the case where saving of an entry value of an entry item regarding an reading report is instructed, the processing circuitry 1 displays the extracted item displaying window in which an unentered item and/or an inconsistent item are/is extracted and listed. This makes it possible to inform a reader that an entry contains an error and to encourage him or her to make a correct entry. For example, accidental omission of entry of a value in a mandatory entry item can be reduced by informing a reader of the presence of an unentered item via the extracted item displaying window. Entry of values inconsistent between the entry items can be reduced by informing the reader of the presence of inconsistent items via the extracted item displaying window.

### Application Example 1

For the same examination on the same patient, not only first reading but also higher-order reading such as second reading, third reading, etc., are conducted. In the reading report displaying screen 30, as shown in FIG. 4, a tab for the first reading and a tab for the second or higher-order reading are displayed. In the case where the tab for the first reading is pressed, a reading report on the first reading is displayed in the current examination displaying area 32. In the case where the tab for the second reading is pressed, a reading report on the second reading is displayed in the current examination displaying area 32. In the case where a reading report on the second reading is not created, the handwriting entry screen 20 for the second reading is displayed on the tablet terminal 6. The reading report on the second reading is created through a similar process to that of the reading report on the first reading. For example, a reader of the second reading performs an entry operation of values in the finding items and the comprehensive evaluation items on the second reading along the flow shown in FIG. 5. In the entry operation, the reader of the second reading may press a copy button 321 in order to enter the entry values in the finding items and the comprehensive evaluation items regarding the first reading in the corresponding finding items and comprehensive evaluation items regarding the second reading.

At the time of higher-order reading, the processing circuitry 1 may judge consistency in the same entry items between the higher-order reading and the first reading by implementing the extracting function 14. Specifically, in the memory 2, the entry item which requires consistency between the first reading and the second reading, and the consistency fulfillment condition are registered in such a manner that they are correlated with each other. In the case where a processing item to be judged is registered in the consistency judgment table, the processing circuitry 1 reads an entry value of the same processing item in the first reading. The processing circuitry 1 reads the consistency fulfillment condition that is correlated with the aforementioned processing item in the consistency judgment table. The processing circuitry 1 then judges whether or not an entry value of the item to be judged for the first reading and an entry value of a corresponding item for the second reading fulfill the consistency fulfillment condition. In the case where these entry values fulfill the consistency fulfillment condition, the item to be judged is not extracted as an inconsistent item. In the case where these entry values do not fulfill the consistency fulfillment condition, the item to be judged is extracted as an inconsistent item.

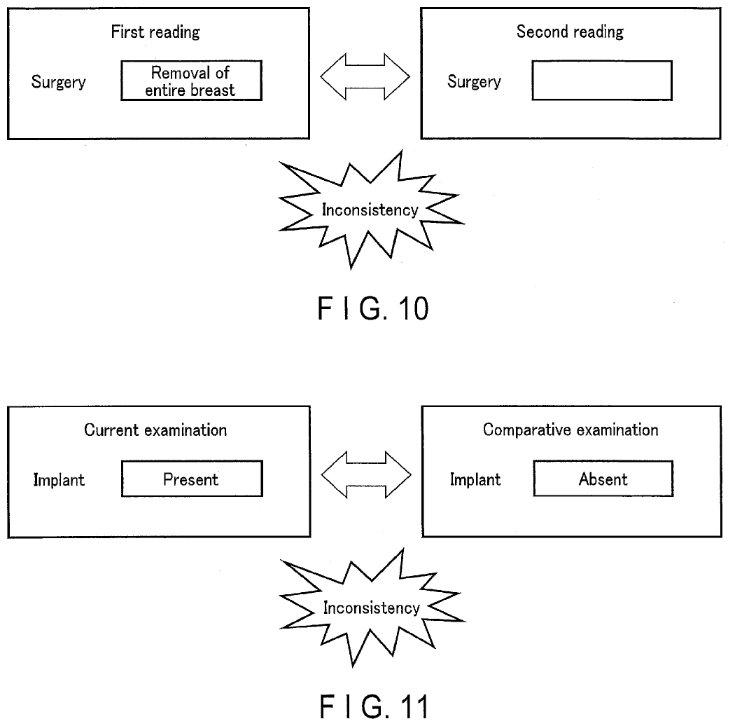

FIG. 10 is a diagram schematically showing processing of judging consistency between the first reading and the second reading. FIG. 10 shows, as an example, the processing of determining consistency regarding the entry item “surgery”. A value of the entry item “surgery” is required to be consistent between the first reading and the second reading. This case assumes that the entry item “surgery” and the consistency fulfillment condition “the same value” are registered in the consistency judgment table in such a manner that this entry item and consistency fulfillment condition are correlated with each other. As shown in FIG. 10, in the case where in spite of the entry item “surgery” for the first reading having a value indicative of “removal of entire breast”, a value of the entry item “surgery” for the second reading is blank, the entry item “surgery” for the second reading may be extracted as an inconsistent item. The extracted entry item “surgery” for the second reading is displayed in the extracted item displaying window along with the entry item “surgery” for the first reading. In this manner, a reader can notice that an entry in the entry item “surgery” for the second reading is erroneous and enter a correct value.

According to the application example 1, by judging consistency in an entry item between the first reading and the second reading, consistency can be improved between the first reading and the second reading.

### Application Example 2

With respect to a current examination, there may be a past examination. In the reading report displaying screen 30, as shown in FIG. 4, a plurality of past examinations correlated with the current examination are displayed in the past examination list 331. From among the plurality of past examinations, a comparative examination is selected via the entry interface 3. For example, from among the plurality of past examinations, the most recent past examination is selected as a comparative examination. A mammography image from the selected comparative examination is displayed on the display 5, etc., and a mammography image from the current examination and the mammography image from the comparative examination are used for comparative reading. A reader performs comparative reading between the mammography image from the current examination and the mammography image from the comparative examination, and performs an entry operation on the finding items and the comprehensive evaluation items regarding a reading report of the current examination along a flow shown in FIG. 5.

When creating a reading report on the current examination, the processing circuitry 1 may judge consistency between the current examination and the comparative examination, with respect to a particular entry item which requires consistency between the current examination and the comparative examination. Specifically, in the memory 2, the entry item which requires consistency between the current examination and the comparative examination, and the consistency fulfillment condition are registered in such a manner that they are correlated with each other. In the case where a processing item to be judged is registered in the consistency judgment table, the processing circuitry 1 reads an entry value of the same processing item for the comparative examination. The processing circuitry 1 reads the consistency fulfillment condition that is correlated with the aforementioned processing item in the consistency judgment table. The processing circuitry 1 then judges whether or not an entry value of the item to be judged in the current examination and an entry value of a corresponding item in the comparative examination fulfill the consistency fulfillment condition. In the case where these entry values fulfill the consistency fulfillment condition, the item to be judged is not extracted as an inconsistent item. In the case where these entry values do not fulfill the consistency fulfillment condition, the item to be judged is extracted as an inconsistent item.

FIG. 11 is a diagram schematically showing processing of judging consistency between a current examination and a comparative examination. FIG. 11 shows, as an example, the processing of determining consistency regarding the entry item “implant”. A value of the entry item “implant” is required to be consistent between the current examination and the comparative examination. This case assumes that the entry item “implant” and the consistency fulfillment condition “the same value” are registered in the consistency judgment table in such a manner that this entry item and consistency fulfillment condition are correlated with each other. As shown in FIG. 11, in the case where a value of the entry item “implant” in the comparative examination indicates “absent” in spite of the fact that the entry item “implant” in the current examination indicates “present”, the processing circuitry 1 can extract the entry item “implant” in the current examination as an inconsistent item. The extracted entry item “implant” in the current examination is displayed in the extracted item displaying window along with the entry item “implant” for the comparative examination. This enables a reader to notice that the entry item “implant” in the current examination is erroneous and to enter a correct value.

According to the application example 2, by judging consistency in entry items between the current examination and the comparative examination, consistency between the current examination and the comparative examination can be improved.

### Application Example 3

In the various embodiments described in the above, values are manually entered in the entry items in steps S2 and S10. However, values may be automatically entered using the copying function 12. For example, as shown in FIG. 4, the copy button 333 is displayed in the reading report displaying screen 30. In the case where the copy button 333 is pressed, the processing circuitry 1 implements the copying function 12, thereby copying an entry value of an entry item in a past examination to the same input item in a current examination. Copying of a value reduces an entry operation in a current examination.

However, regarding the entry item “comparative reading”, if an entry value “absent” of the entry item in the past examination is copied to the entry item in the current examination in spite of the fact that the comparative reading has been performed in the current examination, an erroneous reading report is created. Thus, if a copy instruction is given, the processing circuitry 1 according to the application example 3 searches for a past image and automatically enters, if the past image is present, a value “present” in the entry item “comparative reading” in the current examination even if an entry value of the input item “comparative reading” in the past examination indicates “absent”.

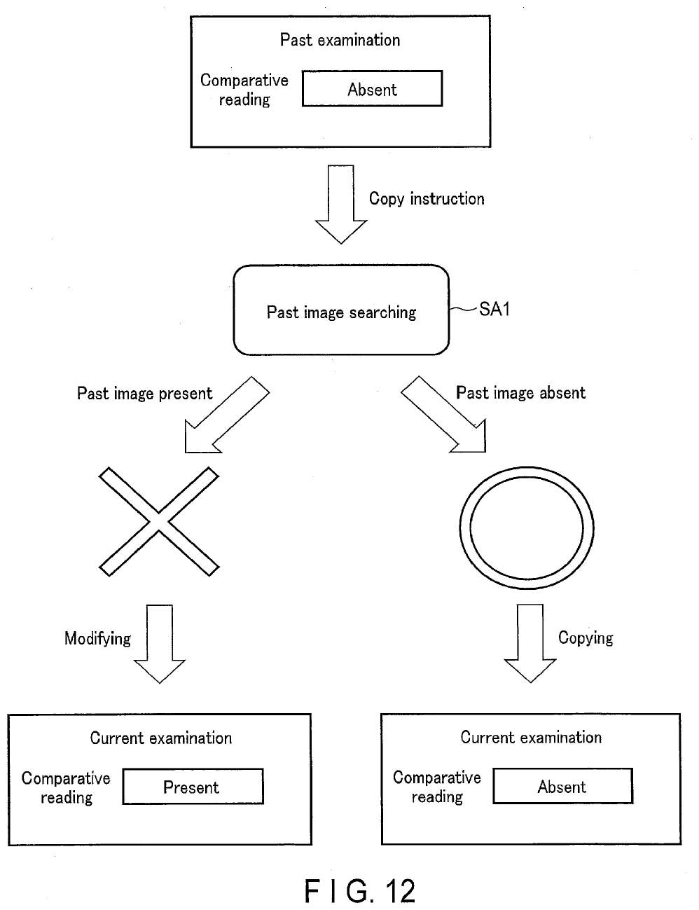

FIG. 12 is a diagram schematically showing an example of processing according to the application examination 3. When a copy instruction is given by, for example, the copy button 333 shown in FIG. 4 being pressed, the processing circuitry 1 reads entry values of respective entry items in the past examination. At this time, as shown in FIG. 12, in the case where the entry value “absent” was entered in the entry item “comparative reading” in the past examination, the processing circuitry 1 implements the extracting function 14, thereby performing past image searching (step SA1). In step SA1, the processing circuitry 1 searches for a past image correlated with the current examination. For example, the processing circuitry 1 judges whether an reading report on a comparative examination correlated with an reading report on a past examination is correlated with a medical image (past image) or not.

In the case where the past image is present, it is assumed that the value “absent” of the entry item “comparative reading” in the current examination is erroneous and the value “present” is correct. The entry item “comparative reading” is extracted as an inconsistent item by the processing circuitry 1. In this case, the processing circuitry 1 modifies the entry value “absent” in the past examination and enters the value “present” in the entry item “comparative reading” in the current examination. When displaying the modified value “present”, the processing circuitry 1 preferably displays this value by emphasizing it with color, etc., in order to notify a reader of the fact that the value was modified from the past examination. In the case where the past image is absent, it is assumed that the value “absent” of the entry item “comparative reading” in the current examination is correct. Therefore, the value “absent” in the past examination is copied to the entry item “comparative reading” in the current examination.

In the case where the value “present” is entered in the entry item “comparative reading” in the current examination, it is necessary to enter one of the values “stable”, “exacerbation”, and “remission” in the entry item “temporal change”. According to the embodiments described above, even if saving is instructed in spite of the fact that no value has been entered in the entry item “temporal change”, the entry item “temporal change” can be extracted as an unentered item or an inconsistent item and be displayed on the extracted item displaying window. In this manner, even if a reader carelessly gives a saving instruction, it is possible to reduce accidental omission of entry of a value with respect to the entry item “temporal change”.

An entry item to which the processing according to the application example 3 is applicable is not limited to “comparative reading” only. Other than the entry item “comparative reading”, the processing according to the application example 3 is applicable to an entry item whose entry value depends on the presence or absence of a past image.

A copying range with the use of the copy button 333, etc., is settable at will. For example, entry values of all entry items in a past examination may be copied to corresponding items in a current examination, or an entry value or entry values of a portion of the entry items in a past examination may be copied to a corresponding item or corresponding items in a current examination. An entry value or entry values of a portion of the entry items can be set by, e.g., a reader at will. Examples of an entry value or entry values of a portion of the entry items may include an annotation that is entered by handwriting on a schema and indicates a lesion site, an entry value of a finding item (for example, a finding attribute and a category) other than the annotation from among the finding items, all of the finding items, all of the total evaluation items, or a portion of the total evaluation items. As a copying range, a given range may be selected via the entry interface 3 or the tablet terminal 6 in accordance with a preference of a reader, a hospital department, a hospital, etc. This realizes improvement in efficiency of creation of a reading report.

According to the application example 3, in the case where an entry value of the entry item “comparative reading” regarding a past examination indicates “absent” and a current examination is correlated with a medical image regarding the past examination, the processing circuitry 1 enters “present” as an entry value in the entry item “comparative reading” regarding the current examination when a copy instruction is given. The above processing realizes both reduction in erroneous entries associated with the use of copying and improvement in working efficiency of a reading report.

### Application Example 4

Regarding the entry item “comparative reading”, there is a case in which, in spite of the fact that a comparative image is absent, an entry value “present” is entered in the entry item “comparative reading” and an entry value “stable” is entered in the entry item “temporal change”. The processing circuitry 1 according to the application example 4 searches for a past image if the entry item “comparative reading” has the entry value “present” and the entry item “temporal change” has the entry value “stable”, and issues a warning when a past image is present.

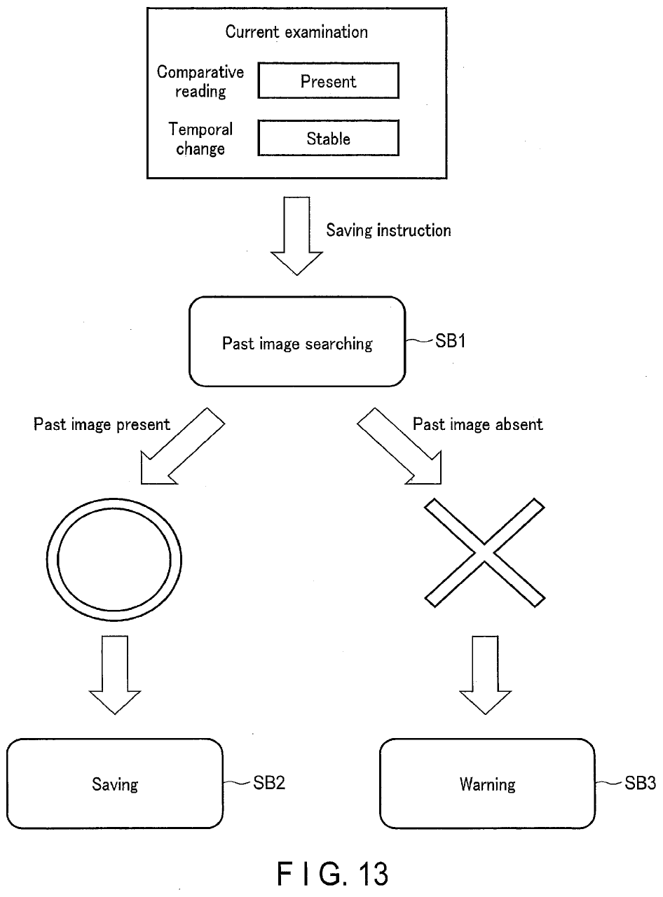

FIG. 13 is a diagram schematically showing an example of processing according to the application example 4. As shown in FIG. 13, assume that the entry value “present” is entered in the entry item “comparative reading”, the entry value “stable” is entered in the entry item “temporal change”, and a saving instruction is given using, e.g., the fix/close button 233 shown in FIG. 3. When saving is instructed, the processing circuitry 1 implements the extracting function 14, thereby performing past image searching (step SB1). In step SB1, the processing circuitry 1 searches for a past image correlated with the current examination. For example, the processing circuitry 1 judges whether an reading report on a comparative examination correlated with an reading report on a past examination is correlated with a medical image (past image) or not.

In the case where the past image is present, it is assumed that the value “stable” of the entry item “temporal change” in the current examination is correct. Accordingly, the processing circuitry 1 stores in the memory 2 the entry value “present” of the entry item “comparative reading” and the entry item “stable” of the entry item “temporal change” (step SB2). In the case where the past image is absent, it is assumed that the value “absent” of the entry item “temporal change” is erroneous. The entry item “temporal change” is extracted as an inconsistent item by the processing circuitry 1. In this case, the processing circuitry 1 issues a warning to the display 5 and the tablet terminal 6 (step SB3). For example, the processing circuitry 1 may display, as a warning, the extracted item displaying window in which the entry item “temporal change” is arranged, or display the entry item “temporal change” on the handwriting entry screen 20 with a visual effect such as color, blinking, etc. Furthermore, a message such as “please check temporal change again” may be displayed. This enables a reader to notice that an entry value of the entry item “temporal change” is erroneous.

According to the application example 4, the processing circuitry 1 issues a warning in the case where the entry value of the entry item “comparative reading” regarding a past examination indicates “present”, the entry item “temporal change” indicates “stable”, and a current examination is not correlated with a medical image regarding the past examination. Even without a comparative image, this processing reduces creation of an reading report in which the entry value “present” is entered in the entry item “comparative reading” and the entry value “stable” is entered in the entry item “temporal change”.

According to at least one of the embodiments described in the above, reading reports can be improved in terms of quality and working efficiency.

The term “processor” used in the above description indicates, for example, a circuit such as a CPU, a GPU, or an Application Specific Integrated Circuit (ASIC), and a programmable logic device (for example, a Simple Programmable Logic Device (SPLD), a Complex Programmable Logic Device (CPLD), and a Field Programmable Gate Array (FPGA). The processor implements functions by reading and executing a program stored in a memory circuit. Instead of storing a program in a memory circuit, a program may be directly integrated into a circuit of the processor. In this case, a processor reads and executes a program integrated into a circuit, thereby realizing its functions. The function corresponding to the program may be realized by a combination of logic circuits, not by executing the program. The processors described in connection with the above embodiments are not limited to single-circuit processors; a plurality of independent processors may be integrated into a single processor that implements such functions. In addition, a plurality of structural elements in FIG. 1 may be integrated in one processor to realize the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A reading report creating apparatus comprising a processing circuitry configured to:
instruct saving of entry values of a plurality of entry items contained in an reading report creating screen;
when saving is instructed, extract from the plurality of entry items a first entry item which requires entry of a value and in which a value has not been entered, and/or a second entry item in which an entry value is inconsistent with a specific entry item;
generate a displaying area in which the first entry item and/or the second entry item are/is arranged; and
display the displaying area on a display.

2. The reading report creating apparatus according to claim 1, wherein the processing circuitry generates the displaying area in which the first entry item and another entered entry item that relates to the first entry item are arranged.

3. The reading report creating apparatus according to claim 2, wherein the processing circuitry displays the first entry item and the another entry item in such a manner as to be visually distinguishable from each other.

4. The reading report creating apparatus according to claim 1, further comprising:
a storage device configured to store, for each respective one of the entry items, a specific entry item required to have consistency with the respective one entry item and a consistency fulfillment condition between the respective one entry item and the specific entry item in such a manner that the specific entry item and the consistency fulfillment condition are correlated with each other, wherein
the processing circuitry judges whether or not an entry value of an entry item to be processed of the plurality of entry items and an entry value of the specific entry item correlated with the entry item to be processed fulfill the consistency fulfillment condition, and extracts, if it is judged that the consistency fulfillment condition is not fulfilled, the entry item to be processed as the second entry item.

5. The reading report creating apparatus according to claim 4, wherein the consistency fulfillment condition is that entry values of two preset entry items are consistent with each other.

6. The reading report creating apparatus according to claim 4, wherein the consistency fulfillment condition is that entry values of corresponding entry items are equivalent to each other between a first reading and a higher-order reading that is performed after the first reading.

7. The reading report creating apparatus according to claim 4, wherein the consistency fulfillment condition is that entry values of corresponding entry items are equivalent to each other between a current examination and a past examination.

8. The reading report creating apparatus according to claim 1, wherein when a copy instruction is given, the processing circuitry copies an entry value of a predetermined entry item regarding a past examination to the predetermined entry item of the plurality of entry items regarding a current examination.

9. The reading report creating apparatus according to claim 8, wherein:
an entry value of the predetermined entry item is an entry value of a comparative reading item regarding a presence or absence of comparative reading; and
in a case where an entry value of the comparative reading item regarding a past examination indicates “absent” and the current examination is correlated with a medical image regarding the past examination, the processing circuitry enters “present” as an entry value in the comparative reading item regarding the current examination when the copy instruction is given.

10. The reading report creating apparatus according to claim 8, wherein:
an entry value of the predetermined entry item is either entry values of the plurality of entry items or an entry value of a portion of the plurality of entry items; and
the entry value of the portion of the entry items is one of an annotation with respect to a schema item of the plurality of entry items, an entry value of a finding item other than the schema item, an entry value with respect to a finding attribute, and an entry value with respect to a category.

11. The reading report creating apparatus according to claim 1, wherein:
the plurality of entry items include a comparative reading item and a temporal change item; and
the processing circuitry issues a warning in a case where an entry value of the comparative reading item regarding a past examination is “present”, the temporal change item is “stable”, and the current examination is not correlated with a medical image regarding the past examination.

12. The reading report creating apparatus according to claim 1, wherein:
the reading report creating screen includes a finding entry area and a comprehensive evaluation entry area; and
the processing circuitry is configured to:
individually instruct the finding entry area and the comprehensive evaluation entry area to perform saving;
extract the first entry item and/or the second entry item for each of the finding entry area and the comprehensive evaluation entry area;
generate the displaying area for each of the finding entry area and the comprehensive evaluation entry area; and
sequentially display the display area for each of the finding entry area and the comprehensive evaluation entry area.
